SEQUENCE LISTING (56 sequences)

SEQ ID NO: 1 - protein, 29 aa, Homo sapiens
HSQGTFTSDYSKYLDSRRAQDFVQWLMNT

SEQ ID NO: 2 - protein, 8 aa, Homo sapiens
KRNRNNIA

SEQ ID NO: 3 - protein, 37 aa, Homo sapiens
HSQGTFTSDYSKYLDSRRAQDFVQWLMNTKRNRNNIA

SEQ ID NO: 4 - protein, 29 aa, Artificial sequence
  Synthetic sequence Formula I of PCT/EP2012/076137
HXXGTFTSDYSXYLDXRRAXDFIXWLXXX

SEQ ID NO: 5 - protein, 29 aa, Artificial sequence
  Synthetic sequence Formula Ia of PCT/EP2012/ 076137
HXQGTFTSDYSKYLDXRRAXDFIXWLXXA

SEQ ID NO: 6 - protein, 29 aa, Artificial sequence
  Synthetic peptide
HXQGTFTSDYSKYLDKRRAKDFIEWLLSA

SEQ ID NO: 7 - protein, 29 aa, Artificial sequence
  Synthetic peptide
HXQGTFTSDYSKYLDERRAKDFIEWLLSA

SEQ ID NO: 8 - protein, 29 aa, Artificial sequence
  Synthetic peptide
HXQGTFTSDYSKYLDERRAKDFIKWLLSA

SEQ ID NO: 9 - protein, 29 aa, Artificial sequence
  Synthetic peptide
HSQGTFTSDYSKYLDERRAKDFIKWLLSA

SEQ ID NO: 10 - protein, 29 aa, Artificial sequence
  Synthetic peptide
HXQGTFTSDYSKYLDERRAKDFIEWLKSA

SEQ ID NO: 11 - protein, 29 aa, Artificial sequence
  Synthetic peptide
HXQGTFTSDYSKYLDERRAKDFIEWLLKA

SEQ ID NO: 12 - protein, 29 aa, Artificial sequence
  Synthetic peptide Compound 1 of PCT/EP2012/ 076137
HXQGTFTSDYSKYLDKRRAKDFIEWLLSA

SEQ ID NO: 13 - protein, 29 aa, Artificial sequence
  Synthetic peptide Compound 2 of PCT/EP2012/ 076137
HXQGTFTSDYSKYLDERRAKDFIEWLLSA

SEQ ID NO: 14 - protein, 29 aa, Artificial sequence
  Synthetic peptide Compound 3 of PCT/EP2012/ 076137
HXQGTFTSDYSKYLDERRAKDFIKWLLSA

SEQ ID NO: 15 - protein, 29 aa, Artificial sequence
  Synthetic peptide Compound 4 of PCT/EP2012/ 076137
HSQGTFTSDYSKYLDERRAKDFIKWLLSA

SEQ ID NO: 16 - protein, 29 aa, Artificial sequence
  Synthetic peptide Compound 5 of PCT/EP2012/ 076137
HXQGTFTSDYSKYLDERRAKDFIEWLKSA

SEQ ID NO: 17 - protein, 29 aa, Artificial sequence
  Synthetic peptide Compound 6 of PCT/EP2012/ 076137
HXQGTFTSDYSKYLDERRAKDFIEWLLKA

SEQ ID NO: 18 - protein, 29 aa, Artificial sequence
  Synthetic sequence Formula II of PCT/EP2012/ 076137
HXXGTFTSDYSXYLDXXRAXDFIXWLXXX

SEQ ID NO: 19 - protein, 29 aa, Artificial sequence
  Synthetic sequence Formula IIa of PCT/EP2012/ 076137
HXXGTFTSDYSRYLDXXRAXDFIXWLLSA

SEQ ID NO: 20 - protein, 29 aa, Artificial sequence
  Synthetic sequence Formula IIb of PCT/EP2012/ 076137
HXXGTFTSDYSRYLDEXRARDFIEWLLSA

SEQ ID NO: 21 - protein, 29 aa, Artificial sequence
  Synthetic peptide
HSQGTFTSDYSRYLDEKRARDFIEWLLSA

SEQ ID NO: 22 - protein, 29 aa, Artificial sequence
  Synthetic peptide
HXQGTFTSDYSRYLDEKRARDFIEWLLSA

SEQ ID NO: 23 - protein, 29 aa, Artificial sequence
  Synthetic peptide
HXQGTFTSDYSRYLDEKRARDFIEWLLSA

SEQ ID NO: 24 - protein, 29 aa, Artificial sequence
  Synthetic peptide
HSHGTFTSDYSRYLDEKRARDFIEWLLSA

SEQ ID NO: 25 - protein, 29 aa, Artificial sequence
  Synthetic peptide
HXHGTFTSDYSRYLDEKRARDFIEWLLSA

SEQ ID NO: 26 - protein, 28 aa, Artificial sequence
  Synthetic peptide
HXGTFTSDYSRYLDEKRARDFIEWLLSA

SEQ ID NO: 27 - protein, 29 aa, Artificial sequence
  Synthetic peptide
HSPGTFTSDYSRYLDEKRARDFIEWLLSA

SEQ ID NO: 28 - protein, 29 aa, Artificial sequence
  Synthetic peptide
HXPGTFTSDYSRYLDEKRARDFIEWLLSA

SEQ ID NO: 29 - protein, 29 aa, Artificial sequence
  Synthetic peptide
HXPGTFTSDYSRYLDEKRARDFIEWLLSA

SEQ ID NO: 30 - protein, 29 aa, Artificial sequence
  Synthetic peptide
HXQGTFTSDYSRYLDEKRAKDFIEWLLSA

SEQ ID NO: 31 - protein, 29 aa, Artificial sequence
  Synthetic peptide Compound 7 of PCT/EP2012/ 076137
HSQGTFTSDYSRYLDEKRARDFIEWLLSA

SEQ ID NO: 32 - protein, 29 aa, Artificial sequence
  Synthetic peptide Compound 8 of PCT/EP2012/ 076137
HXQGTFTSDYSRYLDEKRARDFIEWLLSA

SEQ ID NO: 33 - protein, 29 aa, Artificial sequence
  Synthetic peptide Compound 9 of PCT/EP2012/ 076137
HXQGTFTSDYSRYLDEKRARDFIEWLLSA

SEQ ID NO: 34 - protein, 29 aa, Artificial sequence
  Synthetic peptide Compound 10 of PCT/EP2012/ 076137
HSHGTFTSDYSRYLDEKRARDFIEWLLSA

SEQ ID NO: 35 - protein, 29 aa, Artificial sequence
  Synthetic peptide Compound 11 of PCT/EP2012/ 076137
HXHGTFTSDYSRYLDEKRARDFIEWLLSA

SEQ ID NO: 36 - protein, 29 aa, Artificial sequence
  Synthetic peptide Compound 12 of PCT/EP2012/ 076137
HXHGTFTSDYSRYLDEKRARDFIEWLLSA

SEQ ID NO: 37 - protein, 29 aa, Artificial sequence
  Synthetic peptide Compound 13 of PCT/EP2012/ 076137
HSPGTFTSDYSRYLDEKRARDFIEWLLSA

SEQ ID NO: 38 - protein, 29 aa, Artificial sequence
  Synthetic peptide Compound 14 of PCT/EP2012/ 076137
HXPGTFTSDYSRYLDEKRARDFIEWLLSA

SEQ ID NO: 39 - protein, 29 aa, Artificial sequence
  Synthetic peptide Compound 15 of PCT/EP2012/ 076137
HXPGTFTSDYSRYLDEKRARDFIEWLLSA

SEQ ID NO: 40 - protein, 29 aa, Artificial sequence
  Synthetic peptide Compound 16 of PCT/EP2012/ 076137
HXQGTFTSDYSRYLDEKRAKDFIEWLLSA

SEQ ID NO: 41 - protein, 29 aa, Artificial sequence
  Synthetic sequence Formula III of PCT/EP2012/ 076137
HXXGTFTSDYSXYLDXXRAXDFIXWLXXX

SEQ ID NO: 42 - protein, 29 aa, Artificial sequence
  Synthetic sequence Formula IIIa of PCT/EP2012/ 076137
HXXGTFTSDYSXYLDXXRAXDFIXWLLSA

SEQ ID NO: 43 - protein, 29 aa, Artificial sequence
  Synthetic sequence Formula IIIb of PCT/EP2012/ 076137
HXXGTFTSDYSKYLDEXRAKDFIEWLLSA

SEQ ID NO: 44 - protein, 29 aa, Artificial sequence
  Synthetic peptide
HXQGTFTSDYSKYLDEKRAKDFIEWLLSA

SEQ ID NO: 45 - protein, 29 aa, Artificial sequence
  Synthetic peptide
HSHGTFTSDYSKYLDEKRAKDFIEWLLSA

SEQ ID NO: 46 - protein, 29 aa, Artificial sequence
  Synthetic peptide
HXHGTFTSDYSKYLDEKRAKDFIEWLLSA

SEQ ID NO: 47 - protein, 29 aa, Artificial sequence
  Synthetic peptide
HSPGTFTSDYSKYLDEKRAKDFIEWLLSA

SEQ ID NO: 48 - protein, 29 aa, Artificial sequence
  Synthetic peptide
HXPGTFTSDYSKYLDEKRAKDFIEWLLSA

SEQ ID NO: 49 - protein, 29 aa, Artificial sequence
  Synthetic peptide Compound 17 of PCT/EP2012/ 076137
HXQGTFTSDYSKYLDEKRAKDFIEWLLSA

SEQ ID NO: 50 - protein, 29 aa, Artificial sequence
  Synthetic peptide Compound 18 of PCT/EP2012/ 076137
HSHGTFTSDYSKYLDEKRAKDFIEWLLSA

SEQ ID NO: 51 - protein, 29 aa, Artificial sequence
  Synthetic peptide Compound 19 of PCT/EP2012/ 076137
HXHGTFTSDYSKYLDEKRAKDFIEWLLSA

SEQ ID NO: 52 - protein, 30 aa, Artificial sequence
  Synthetic peptide Compound 20 of PCT/EP2012/ 076137
HHSPGTFTSDYSKYLDEKRAKDFIEWLLSA

SEQ ID NO: 53 - protein, 29 aa, Artificial sequence
  Synthetic peptide Compound 21 of PCT/EP2012/ 076137
HXPGTFTSDYSKYLDEKRAKDFIEWLLSA

SEQ ID NO: 54 - protein, 29 aa, Artificial sequence
  Synthetic peptide Compound 22 of PCT/EP2012/ 076137
HXQGTFTSDYSKYLDEKRAKDFIEWLLSA

SEQ ID NO: 55 - protein, 29 aa, Artificial sequence
  Synthetic peptide Compound 23 of PCT/EP2012/ 076137
HXQGTFTSDYSKYLDEKRAKDFIEWLLSA

SEQ ID NO: 56 - protein, 29 aa, Artificial sequence
  Synthetic peptide Compound 24 of PCT/EP2012/ 076137
HXQGTFTSDYSKYLDEKRAKDFIEWLLSA

(19) United States
(12) Patent Application Publication
(10) Pub. No.: US 2013/0316941 A1
(43) Pub. Date: Nov. 28, 2013

(54) GLUCAGON ANALOGUES

(71) Applicants: Boehringer Ingelheim International GmbH, Ingelheim am Rhein (DE); Zealand Pharma A/S, Glostrup (DK)

(72) Inventors: Dieter Wolfgang Hamprecht, Ingelheim am Rhein (DE); Jakob Lind Tolborg, Herlev (DK); Ditte Riber, Bronshoj (DK)

(73) Assignees: Boehringer Ingelheim International GmbH, Ingelheim am Rhein (DE); Zealand Pharma A/S, Glostrup (DK)

(21) Appl. No.: 13/720,041

(22) Filed: Dec. 19, 2012

Related U.S. Application Data
(60) Provisional application No. 61/579,888, filed on Dec. 23, 2011.

(51) Int. Cl. C07K 14/605; A61K 45/06; A61K 38/26

(52) U.S. Cl. CPC C07K 14/605; A61K 38/26; A61K 45/06
USPC 514/1.9; 530/308; 514/11.7; 536/23.51; 514/5.3; 514/7.4; 514/7.2; 514/6.5; 435/320.1; 435/369

(57) ABSTRACT

The invention provides glucagon analogue peptides and their use for promoting weight loss or preventing weight gain, and the treatment of obesity or excess body weight and associated conditions. The compounds may also be used to improve glycemic control and/or for the treatment of diabetes. The compounds may mediate their effect, inter alia, by having increased selectivity for the GLP-1 receptor as compared to human glucagon.

### CROSS-REFERENCE TO RELATED APPLICATIONS

This application claims benefit of U.S. Provisional Application No. 61/579,888, filed Dec. 23, 2011, which is hereby incorporated by reference.

### FIELD OF THE INVENTION

The present invention relates to glucagon analogues and their medical use, for example in the treatment of excess food intake, obesity and excess weight and associated conditions, and elevated cholesterol. The compounds may also be used to improve glycaemic control and/or for the treatment of diabetes.

### BACKGROUND OF THE INVENTION

Preproglucagon is a 158 amino acid precursor polypeptide that is differentially processed in the tissues to form a number of structurally related proglucagon-derived peptides, including glucagon (Glu), glucagon-like peptide-1 (GLP-1), glucagon-like peptide-2 (GLP-2), and oxyntomodulin (OXM). These molecules are involved in a wide variety of physiological functions, including glucose homeostasis, insulin secretion, gastric emptying and intestinal growth, as well as regulation of food intake.

Glucagon is a 29-amino acid peptide that corresponds to amino acids 53 to 81 of pre-proglucagon and has the sequence His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr. Oxyntomodulin (OXM) is a 37 amino acid peptide which includes the complete 29 amino acid sequence of glucagon with an octapeptide carboxyterminal extension (amino acids 82 to 89 of pre-proglucagon, having the sequence Lys-Arg-Asn-Arg-Asn-Asn-Ile-Ala and termed “intervening peptide 1” or IP-1; the full sequence of human oxyntomodulin is thus His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-Lys-Arg-Asn-Arg-Asn-Asn-Ile-Ala). The major biologically active fragment of GLP-1 is produced as a 30-amino acid, C-terminally amidated peptide that corresponds to amino acids 98 to 127 of pre-proglucagon.

Glucagon helps maintain the level of glucose in the blood by binding to glucagon receptors on hepatocytes, causing the liver to release glucose—stored in the form of glycogen—through glycogenolysis. As these stores become depleted, glucagon stimulates the liver to synthesize additional glucose by gluconeogenesis. This glucose is released into the bloodstream, preventing the development of hypoglycemia.

OXM is released into the blood in response to food ingestion and in proportion to meal calorie content. OXM has been shown to suppress appetite and inhibit food intake in humans (Cohen et al, Journal of Endocrinology and Metabolism, 88, 4696-4701, 2003; WO 2003/022304). In addition to those anorectic effects, which are similar to those of GLP-1, OXM must also affect body weight by another mechanism, since rats treated with oxyntomodulin show less body weight gain than pair-fed rats (Bloom, Endocrinology 2004, 145, 2687). Treatment of obese rodents with OXM also improves their glucose tolerance (Parlevliet et al, Am J Physiol Endocrinol Metab, 294, E142-7, 2008) and suppresses body weight gain (WO 2003/022304).

OXM activates both the glucagon and the GLP-1 receptors with a two-fold higher potency for the glucagon receptor over the GLP-1 receptor, but is less potent than native glucagon and GLP-1 on their respective receptors. Human glucagon is also capable of activating both receptors, though with a strong preference for the glucagon receptor over the GLP-1 receptor. GLP-1 on the other hand is not capable of activating glucagon receptors. The mechanism of action of oxyntomodulin is not well understood. In particular, it is not known whether some of the extrahepatic effects of the hormone are mediated through the GLP-1 and glucagon receptors, or through one or more unidentified receptors.

Other peptides have been shown to bind and activate both the glucagon and the GLP-1 receptor (Hjort et al, Journal of Biological Chemistry, 269, 30121-30124, 1994) and to suppress body weight gain and reduce food intake (WO 2006/134340, WO 2007/100535, WO 2008/10101, WO 2008/152403, WO 2009/155257 and WO 2009/155258).

Diabetes, especially type 2 diabetes, is establishing itself as an epidemic of the 21^{st} century with an estimated 5% of the adult world population suffering from the disease. The number of deaths attributable to diabetes is steadily growing, currently estimated at 3.8 million cases each year, reflecting the insufficient glycaemic control achieved with currently available treatments. Therefore, more effective therapeutics for glycaemic control are needed.

Obesity is a globally increasing health problem is associated with various diseases, particularly cardiovascular disease (CVD), type 2 diabetes, obstructive sleep apnea, certain types of cancer, and osteoarthritis. As a result, obesity has been found to reduce life expectancy. According to 2005 projections by the World Health Organization there are 400 million adults (age >15) classified as obese worldwide. In the US, obesity is now believed to be the second-leading cause of preventable death after smoking.

The rise in obesity drives an increase in diabetes, and approximately 90% of people with type 2 diabetes may be classified as obese. There are 246 million people worldwide with diabetes, and by 2025 it is estimated that 380 million will have diabetes. Many have additional cardiovascular risk factors, including high/aberrant LDL and triglycerides and low HDL.

Further conditions are associated with metabolic diseases, e.g. hypertension, atherogenic dyslipidemia, atherosclerosis, coronary heart disease, stroke and obesity linked inflammation. Accordingly, a treatment for the underlying metabolic disease might have a positive impact on follow-on conditions.

Accordingly, there is a strong medical need for treating metabolic and associated diseases such as obesity, dyslipidemia and diabetes.

### SUMMARY OF THE INVENTION

In a first aspect, the invention provides a compound having the formula:

R¹—X—Z—R²

wherein

R¹ is H, C₁₋₄ alkyl, acetyl, formyl, benzoyl or trifluoroacetyl;

R² is OH or NH₂;

X is a peptide which has the formula I:

His-X2-X3-Gly-Thr-Phe-Thr-Ser-Asp-Tyr-Ser-X12-Tyr-Leu-Asp-X16-Arg-Arg-Ala-X20-Asp-Phe-Ile-
X24-Trp-Leu-X27-X28-X29 (I)

wherein

X2 is selected from Ser, D-Ser and Aib;

X3 is selected from Gln, His and Pro;

X12 is selected from Lys and Y

X16 is selected from Glu and Y;

X20 is selected from Lys and Y;

X24 is selected from Glu and Y;

X27 is selected from Leu and Y;

X28 is selected from Ser and Y or is absent;

X29 is Ala or absent;

wherein at least one of X12, X16, X17, X20, X27 and X28 is Y;
wherein each residue Y is independently selected from Lys, Cys and Orn;
wherein the side chain of at least one amino acid residue Y is conjugated to a lipophilic substituent having the formula:
(i) Z¹, wherein Z¹ is a lipophilic moiety conjugated directly to the side chain of Y; or
(ii) Z¹Z², wherein Z¹ is a lipophilic moiety, Z² is a spacer, and Z¹ is conjugated to the side chain of Y via Z²;
and Z is absent or is a sequence of 1-20 amino acid units independently selected from the group consisting of Ala, Leu, Ser, Thr, Tyr, Cys, Glu, Lys, Arg, Dbu, Dpr and Orn;
or a pharmaceutically acceptable salt thereof.

Peptide X may have the formula Ia:

His-X2-Gln-Gly-Thr-Phe-Thr-Ser-Asp-Tyr-Ser-Lys-Tyr-Leu-Asp-X16-Arg-Arg-Ala-X20-Asp-Phe-Ile-
X24-Trp-Leu-X27-X28-Ala (Ia)

wherein

X2 is selected from Ser, D-Ser and Aib;

X16 is selected from Glu and Y;

X20 is selected from Lys and Y;

X24 is selected from Glu and Y;

X27 is selected from Leu and Y; and

X28 is selected from Ser and Y.

Peptide X may have the sequence:

H-Aib-QGTFTSDYSKYLDKRRAKDFIEWLLSA;
H-Aib-QGTFTSDYSKYLDERRAKDFIEWLLSA;
H-Aib-QGTFTSDYSKYLDERRAKDFIKWLLSA;
HSQGTFTSDYSKYLDERRAKDFIKWLLSA;
H-Aib-QGTFTSDYSKYLDERRAKDFIEWLKSA;
or
H-Aib-QGTFTSDYSKYLDERRAKDFIEWLLKA.

For example, peptide X may be:

H-Aib-QGTFTSDYSKYLDK*RRAKDFIEWLLSA;
H-Aib-QGTFTSDYSKYLDERRAK*DFIEWLLSA;
H-Aib-QGTFTSDYSKYLDERRAKDFIK*WLLSA;
HSQGTFTSDYSKYLDERRAKDFIK*WLLSA;
H-Aib-QGTFTSDYSKYLDERRAKDFIEWLK*SA;
or
H-Aib-QGTFTSDYSKYLDERRAKDFIEWLLK*A;

wherein K* indicates a Lys residue to which the lipophilic substituent is conjugated.

For example, the compound may be:

H-H-Aib-QGTFTSDYSKYLD-K(Hexadecanoyl-isoGlu)-RRAKDFIEWLLSA-NH₂ [Compound 1];
H-H-Aib-QGTFTSDYSKYLD ER RA-K(Hexadecanoyl-isoGlu)-DFIEWLLSA-NH₂ [Compound 2];
H-H-Aib-QGTFTSDYSKYLDERRAKDFI-K(Hexadecanoyl-isoGlu)-WLLSA-NH₂ [Compound 3];
H-HSQGTFTSDYSKYLDERRAKDFI-K(Hexadecanoyl-isoGlu)-WLLSA-NH₂ [Compound 4];
H-H-Aib-QGTFTSDYSKYLDERRAKDFIEWL-K(Hexadecanoyl-isoGlu)-SA-NH₂ [Compound 5];
or
H-H-Aib-QGTFTSDYSKYLDERRAKDFIEWLL-K(Hexadecanoyl-isoGlu)-A-NH₂ [Compound 6];
or a pharmaceutically acceptable salt thereof.

In a second aspect, the invention provides a compound having the formula

R¹—X—Z—R²

wherein

R¹ is H, C₁₋₄ alkyl, acetyl, formyl, benzoyl or trifluoroacetyl;

R² is OH or NH₂;

X is a peptide which has the formula II:

His-X2-X3-Gly-Thr-Phe-Thr-Ser-Asp-Tyr-Ser-X12-Tyr-Leu-Asp-X16-X17-Arg-Ala-X20-Asp-Phe-Ile-
X24-Trp-Leu-X27-X28-X29 (II)

wherein

X2 is selected from Ser, D-Ser and Aib;

X3 is selected from Gln, His and Pro;

X12 is selected from Arg, Lys and Y;

X16 is selected from Glu and Y;

X17 is selected from Arg and Y;

X20 is selected from Lys, Arg and Y;

X24 is selected from Glu and Y;

X27 is selected from Leu and Y;

X28 is selected from Ser and Y or absent;

X29 is Ala or absent;

wherein X12 and/or X20 is Arg;
wherein at least one of X12, X16, X17, X20, X24, X27 and X28 is Y;
wherein each residue Y is independently selected from Lys, Cys and Orn;
wherein the side chain of at least one amino acid residue Y is conjugated to a lipophilic substituent having the formula:
(i) Z¹, wherein Z¹ is a lipophilic moiety conjugated directly to the side chain of Y; or
(ii) Z¹Z², wherein Z¹ is a lipophilic moiety, Z² is a spacer, and Z¹ is conjugated to the side chain of Y via Z²;
and Z is absent or is a sequence of 1-20 amino acid units independently selected from the group consisting of Ala, Leu, Ser, Thr, Tyr, Cys, Glu, Lys, Arg, Dbu, Dpr and Orn;
or a pharmaceutically acceptable salt thereof.

It may be desirable that X12 is Arg.

Peptide X may have the formula IIa:

His-X2-X3-Gly-Thr-Phe-Thr-Ser-Asp-Tyr-Ser-Arg-Tyr-Leu-Asp-X16-X17-Arg-Ala-X20-Asp-Phe-Ile-
X24-Trp-Leu-Leu-Ser-Ala (IIa)

wherein

X2 is selected from Ser, D-Ser and Aib;

X3 is selected from Gln, His and Pro;

X16 is selected from Glu and Y;

X17 is selected from Arg and Y;

X20 is selected from Arg and Lys; and

X24 is selected from Glu and Y.

Peptide X may have the formula IIb:

His-X2-X3-Gly-Thr-Phe-Thr-Ser-Asp-Tyr-Ser-Arg-Tyr-Leu-Asp-Glu-X17-Arg-Ala-Arg-Asp-Phe-Ile-
Glu-Trp-Leu-Leu-Ser-Ala (IIb)

wherein

X2 is selected from Ser, D-Ser and Aib;

X3 is selected from Gln, His and Pro; and

X17 is Y.

Peptide X may have the sequence:

HSQGTFTSDYSRYLDEKRARDFIEWLLSA;
H-DSer-QGTFTSDYSRYLDEKRARDFIEWLLSA;
H-Aib-QGTFTSDYSRYLDEKRARDFIEWLLSA;
HSHGTFTSDYSRYLDEKRARDFIEWLLSA;
H-DSer-HGTFTSDYSRYLDEKRARDFIEWLLSA;
H-Aib-GTFTSDYSRYLDEKRARDFIEWLLSA;
HSPGTFTSDYSRYLDEKRARDFIEWLLSA;
H-DSer-PGTFTSDYSRYLDEKRARDFIEWLLSA;
H-Aib-PGTFTSDYSRYLDEKRARDFIEWLLSA;
or
H-Aib-QGTFTSDYSRYLDEKRAKDFIEWLLSA.

For example, X may be:

HSQGTFTSDYSRYLDEK*RARDFIEWLLSA;
H-DSer-QGTFTSDYSRYLDEK*RARDFIEWLLSA;
H-Aib-QGTFTSDYSRYLDEK*RARDFIEWLLSA;
HSHGTFTSDYSRYLDEK*RARDFIEWLLSA;
H-DSer-HGTFTSDYSRYLDEK*RARDFIEWLLSA;
H-Aib-GTFTSDYSRYLDEK*RARDFIEWLLSA;
HSPGTFTSDYSRYLDEK*RARDFIEWLLSA;
H-DSer-PGTFTSDYSRYLDEK*RARDFIEWLLSA;
H-Aib-PGTFTSDYSRYLDEK*RARDFIEWLLSA;
or
H-Aib-QGTFTSDYSRYLDEK*RAKDFIEWLLSA.

wherein K* indicates a Lys residue to which the lipophilic substituent is conjugated.

The compound may be:

H-HSQGTFTSDYSRYLDE-K(Hexadecanoyl-isoGlu)-RARDFIEWLLSA-NH₂ [Compound 7];
H-H-DSer-QGTFTSDYSRYLDE-K(Hexadecanoyl-isoGlu)-RARDFIEWLLSA-NH₂ [Compound 8];
H-H-Aib-QGTFTSDYSRYLDE-K(Hexadecanoyl-isoGlu)-RARDFIEWLLSA-NH₂ [Compound 9];
H-HSHGTFTSDYSRYLDE-K(Hexadecanoyl-isoGlu)-RARDFIEWLLSA-NH₂ [Compound 10];
H-H-DSer-HGTFTSDYSRYLDE-K(Hexadecanoyl-isoGlu)-RARDFIEWLLSA-NH₂ [Compound 11];
H-H-Aib-HGTFTSDYSRYLDE-K(Hexadecanoyl-isoGlu)-RARDFIEWLLSA-NH₂ [Compound 12];
H-HSPGTFTSDYSRYLDE-K(Hexadecanoyl-isoGlu)-RARDFIEWLLSA-NH₂ [Compound 13];
H-H-DSer-PGTFTSDYSRYLDE-K(Hexadecanoyl-isoGlu)-RARDFIEWLLSA-NH₂ [Compound 14];
H-H-Aib-PGTFTSDYSRYLDE-K(Hexadecanoyl-isoGlu)-RARDFIEWLLSA-NH₂ [Compound 15];
or
H-H-Aib-QGTFTSDYSRYLDE-K(Hexadecanoyl-isoGlu)-RAKDFIEWLLSA-NH₂ [Compound 16];

or a pharmaceutically acceptable salt thereof.

In a third aspect, the invention provides a compound having the formula

R¹—X—Z—R²

wherein

R¹ is H, C₁₋₄ alkyl, acetyl, formyl, benzoyl or trifluoroacetyl;

R² is OH or NH₂;

X is a peptide which has the formula III:

His-X2-X3-Gly-Thr-Phe-Thr-Ser-Asp-Tyr-Ser-X12-Tyr-Leu-Asp-X16-X17-Arg-Ala-X20-Asp-Phe-Ile-
X24-Trp-Leu-X27-X28-X29 (III)

wherein

X2 is selected from Ser, D-Ser and Aib;

X3 is selected from Gln, His and Pro;

X12 is selected from Lys and Y

X16 is selected from Glu and Y;

X17 is selected from Arg and Y;

X20 is selected from Lys and Y;

X24 is selected from Glu and Y;

X27 is selected from Leu and Y;

X28 is selected from Ser and Y or is absent;

X29 is Ala or absent;

wherein X3 is His or Pro when X2 is Ser or Aib, and X2 is D-Ser when X3 is Gln;
wherein at least one of X12, X16, X17, X20, X24, X27 and X28 is Y;
wherein each residue Y is independently selected from Lys, Cys and Orn;
wherein the side chain of at least one amino acid residue Y of X is conjugated to a lipophilic substituent having the formula:
(i) Z¹, wherein Z¹ is a lipophilic moiety conjugated directly to the side chain of Y; or
(ii) Z¹Z², wherein Z¹ is a lipophilic moiety, Z² is a spacer, and Z¹ is conjugated to the side chain of Y via Z²;
and Z is absent or is a sequence of 1-20 amino acid units independently selected from the group consisting of Ala, Leu, Ser, Thr, Tyr, Cys, Glu, Lys, Arg, Dbu, Dpr and Orn;
or a pharmaceutically acceptable salt thereof.

Peptide X may have the formula IIIa:

His-X2-X3-Gly-Thr-Phe-Thr-Ser-Asp-Tyr-Ser-X12-Tyr-Leu-Asp-X16-X17-Arg-Ala-X20-Asp-Phe-Ile-
X24-Trp-Leu-Leu-Ser-Ala (IIIa)

wherein

X2 is selected from Ser, D-Ser and Aib;

X3 is selected from Gln, His and Pro;

X12 is selected from Lys and Y

X16 is selected from Glu and Y;

X17 is selected from Arg and Y;

X20 is selected from Lys and Y; and

X24 is selected from Glu and Y.

Peptide X may have the formula IIIb:

His-X2-X3-Gly-Thr-Phe-Thr-Ser-Asp-Tyr-Ser-Lys-Tyr-Leu-Asp-Glu-X17-Arg-Ala-Lys-Asp-Phe-Ile-
Glu-Trp-Leu-Leu-Ser-Ala (IIIb)

wherein

X2 is selected from Ser, D-Ser and Aib;

X3 is selected from Gln, His and Pro; and

X17 is Y.

Peptide X may have the sequence:

H-DSer-QGTFTSDYSKYLDEKRAKDFIEWLLSA;
HSHGTFTSDYSKYLDEKRAKDFIEWLLSA;
H-DSer-HGTFTSDYSKYLDEKRAKDFIEWLLSA;
HSPGTFTSDYSKYLDEKRAKDFIEWLLSA;
or
H-DSer-PGTFTSDYSKYLDEKRAKDFIEWLLSA.

Peptide X may be:

H-DSer-QGTFTSDYSKYLDEK*RAKDFIEWLLSA;
HSHGTFTSDYSKYLDEK*RAKDFIEWLLSA;
H-DSer-HGTFTSDYSKYLDEK*RAKDFIEWLLSA;
HSPGTFTSDYSKYLDEK*RAKDFIEWLLSA;
or
H-DSer-PGTFTSDYSKYLDEK*RAKDFIEWLLSA;

wherein K* indicates a Lys residue to which the lipophilic substituent is conjugated.

The compound may be:

H-H-DSer-QGTFTSDYSKYLDE-K(Hexadecanoyl-isoGlu)-RAKDFIEWLLSA-NH2 [Compound 17];
H-HSHGTFTSDYSKYLDE-K(Hexadecanoyl-isoGlu)-RAKDFIEWLLSA-NH2 [Compound 18];
H-H-DSer-HGTFTSDYSKYLDE-K(Hexadecanoyl-isoGlu)-RAKDFIEWLLSA-NH2 [Compound 19];
H-HSPGTFTSDYSKYLDE-K(Hexadecanoyl-isoGlu)-RAKDFIEWLLSA-NH2 [Compound 20];
or
H-H-DSer-PGTFTSDYSKYLDE-K(Hexadecanoyl-isoGlu)-RAKDFIEWLLSA-NH2 [Compound 21];
or a pharmaceutically acceptable salt thereof.

In any of the above aspects of the invention, it may be desirable that peptide X contains only one residue Y.

Whether peptide X contains one or more than one residue Y, the or each residue Y may be Lys.

The invention further provides an isolated nucleic acid (which may be DNA or RNA) encoding a peptide X—Z as defined in any of the three aspects of the invention described above, i.e. the peptide backbone of any of these compounds of the invention, before addition of the lipophilic substituent to any residue Y. (Of course, this may only be appropriate when each residue in X—Z is one of the 20 naturally occurring amino acids which can be incorporated into protein by nucleic acid translation.) Further provided is an expression vector comprising such a nucleic acid, and a host cell containing such a nucleic acid or expression vector.

In a fourth aspect, the invention provides the compounds:

H-H-Aib-QGTFTSDYSKYLDE-K(Octadecanoyl-isoGlu)-RAKDFIEWLLSA-NH₂ [Compound 22];
H-H-Aib-QGTFTSDYSKYLDE-K(Hexadecanoyl-isoGlu)-RAKDFIEWLLSA-OH [Compound 23];
and
H-H-Aib-QGTFTSDYSKYLDE-K(Octadecanoyl-isoGlu)-RAKDFIEWLLSA-OH [Compound 24].

The present invention further provides a composition comprising a compound, nucleic acid, expression vector or host cell of the invention in admixture with a carrier. In preferred embodiments, the composition is a pharmaceutically acceptable composition and the carrier is a pharmaceutically acceptable carrier. The composition may contain a pharmaceutically acceptable salt of the compound of the invention.

In addition, the present invention provides a compound or composition of any aspect of the invention as described above for use in a method of medical treatment.

The compounds described find use, inter alia, in preventing weight gain or promoting weight loss. By “preventing” is meant inhibiting or reducing when compared to the absence of treatment, and is not necessarily meant to imply complete cessation of weight gain. The peptides may cause a decrease in food intake and/or increased energy expenditure, resulting in the observed effect on body weight.

Independently of their effect on body weight, the compounds of the invention may have a beneficial effect on circulating cholesterol levels, being capable of lowering circulating LDL levels and increasing HDL/LDL ratio.

The compounds may additionally have a beneficial effect on glycaemic control, independently of their effect on body weight. It is envisaged that such compounds may be therapeutically useful in conditions which are not directly associated with or caused by excess weight or obesity, such as type I diabetes and gestational diabetes.

Of course, this does not preclude their use in conditions ultimately caused or exacerbated by obesity or excess weight. Indeed, their effect on glucose control and body weight may make them particularly suitable for treatment of such conditions.

Thus the compounds of the invention can be used for direct or indirect therapy of any condition caused or characterised by excess body weight, such as the treatment and/or prevention of obesity, morbid obesity, obesity linked inflammation, obesity linked gallbladder disease, obesity induced sleep apnea. They may also be used for the prevention of metabolic syndrome, type I diabetes, type II diabetes, hypertension, atherogenic dyslipidemia, atherosclerosis, arteriosclerosis, coronary heart disease, or stroke. Their effects in these conditions may be as a result of or associated with their effect on body weight, or may be independent thereof.

Thus, the invention provides a compound of the invention for use in a method of preventing weight gain or promoting weight loss in an individual in need thereof. Also provided is the use of a compound of the invention in the manufacture of a medicament for preventing weight gain or promoting weight loss in an individual. Also provided is a method of preventing weight gain or promoting weight loss in an individual in need thereof comprising administering a compound of the invention to the individual.

The invention further provides a compound of the invention for use in a method of lowering circulating LDL levels, and/or increasing HDL/LDL ratio in an individual in need thereof. Also provided is the use of a compound of the invention in the manufacture of a medicament for lowering circulating LDL levels, and/or increasing HDL/LDL ratio in an individual. Also provided is a method of lowering circulating LDL levels, and/or increasing HDL/LDL ratio in an individual in need thereof comprising administering a compound of the invention to the individual.

The invention further provides a compound of the invention for use in a method of prevention or treatment of a condition caused or characterised by excess body weight. Also provided is the use of a compound of the invention in the manufacture of a medicament for prevention or treatment of a condition caused or characterised by excess body weight. Also provided is a method of prevention or treatment of a condition caused or characterised by excess body weight in an individual in need thereof comprising administering a compound of the invention to the individual.

The invention further provides a compound of the invention for use in a method of prevention and/or treatment of obesity, morbid obesity, morbid obesity prior to surgery, obesity linked inflammation, obesity linked gallbladder disease, obesity induced sleep apnea, type I diabetes, type II diabetes, metabolic syndrome, hypertension, atherogenic dyslipidemia, atherosclerois, arteriosclerosis, coronary heart disease, peripheral artery disease, stroke or microvascular disease in an individual in need thereof. Also provided is the use of a compound of the invention in the manufacture of a medicament for prevention or treatment of such a condition. Also provided is a method of prevention or treatment of such a condition in an individual in need thereof comprising administering a compound of the invention to the individual.

The invention further provides a compound of the invention for use in conjunction with an agent for treatment of obesity, dyslipidemia, diabetes, or hypertension. Also provided is the use of a compound of the invention in the manufacture of a medicament for use in conjunction with an agent for treatment of obesity, dyslipidemia, diabetes, or hypertension. Also provided is a method of treatment comprising administration of a compound of the invention in conjunction with an agent for treatment of obesity, dyslipidemia, diabetes, or hypertension to an individual in need thereof. Also provided is a pharmaceutical composition comprising a compound of the invention and an agent for treatment of obesity, dyslipidemia, diabetes, or hypertension.

The agent for treatment of obesity may be a glucagon-like peptide receptor 1 agonist, peptide YY receptor agonist or analogue thereof, cannabinoid receptor 1 antagonist, lipase inhibitor, melanocortin receptor 4 agonist, or melanin concentrating hormone receptor 1 antagonist.

The agent for treatment of hypertension may be an angiotensin-converting enzyme inhibitor, angiotensin II receptor blocker, diuretic, beta-blocker, or calcium channel blocker.

The agent for treatment of dyslipidaemia may be a statin, a fibrate, a niacin and/or a cholesterol absorbtion inhibitor.

The agent for treatment of diabetes may be metformin, a sulfonylurea, a glinide, a DPP-IV inhibitor, a glitazone, a GLP-1 agonist, insulin or an insulin analogue.

As already described, the invention extends to expression vectors comprising the above-described nucleic acid sequence, optionally in combination with sequences to direct its expression, and host cells containing the expression vectors. Preferably the host cells are capable of expressing and secreting the compound of the invention, or the peptide backbone X—Z of the compound of the invention. In a still further aspect, the present invention provides a method of producing the compound, the method comprising culturing the host cells under conditions suitable for expressing the compound and purifying the compound thus produced. The method may comprise the further step of adding the lipophilic substituent at the appropriate amino acid position.

The invention further provides a nucleic acid of the invention, an expression vector of the invention, or a host cell capable of expressing and secreting a compound of the invention, for use in a method of medical treatment. It will be understood that the nucleic acid, expression vector and host cells may be used for treatment of any of the disorders described herein which may be treated with the compounds of the invention themselves. References to a therapeutic composition comprising a compound of the invention, administration of a compound of the invention, or any therapeutic use thereof, should therefore be construed to encompass the equivalent use of a nucleic acid, expression vector or host cell of the invention, except where the context demands otherwise.

### DETAILED DESCRIPTION OF THE INVENTION

Throughout this specification, the conventional one letter and three letter codes for naturally occurring amino acids are used, as well as generally accepted three letter codes for other amino acids, such as Aib (α-aminoisobutyric acid), Orn (ornithine), Dbu (2,4 diaminobutyric acid), D-Ser (D-form of Ser) and Dpr (2,3-diaminopropanoic acid).

The term “native glucagon” refers to native human glucagon having the sequence H-His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-OH (SEQ ID NO: 1).

The invention provides compounds as defined above. For the avoidance of doubt, in the definitions provided herein, it is generally intended that the sequence of peptide X can only be varied at those positions which are stated to allow variation, and only within the options stated. Amino acids within the sequence X can be considered to be numbered consecutively from 1 to 29 in the conventional N-terminal to C-terminal direction. Reference to a “position” within X should be construed accordingly, as should reference to positions within native human glucagon and other molecules.

Without wishing to be bound by any particular theory, the residues at positions 27, 28 and 29 of native glucagon appear to provide significant selectivity of the peptide for the glucagon receptor. The residues present at these positions in the compounds of the invention may increase potency at and/or selectivity for the GLP-1 receptor, potentially without significant reduction of potency at the glucagon receptor.

Substitution of the naturally-occurring Met residue at position 27 (e.g. with Leu or Lys, especially with Leu) also reduces the potential for oxidation, thereby increasing the chemical stability of the compounds.

Substitution of the naturally-occurring Asn residue at position 28 (e.g. by Ser, Arg or Ala) also reduces the potential for deamidation in acidic solution, so increasing the chemical stability of the compounds.

Substitution of one or both of the naturally-occurring Gln residues at positions 20 and 24 also reduces the potential for deamidation in acidic solution, thereby increasing the chemical stability of the compounds.

Substitution of one or more of the naturally occurring amino acids at positions 12, 16, 17, 20, 24, 27 and 28 with a suitable amino acid Y enables conjugation to a lipophilic substituent. The residue(s) Y at these positions may independently be Cys, Orn or Lys. More particularly, one or more of these residues may be Cys. Further, one or more of the residues at these positions may be Lys. Where the compound contains more than one residue Y, they may be the same (all Cys, all Orn, or all Lys) or different. In some embodiments it may be desirable that each peptide X contains just one residue Y. The or each residue Y may be Lys.

As already disclosed, a compound of the invention may comprise a C-terminal peptide sequence Z of 1-20 amino acids, for example to stabilise the conformation and/or secondary structure of the glucagon analogue peptide, and/or to render the glucagon analogue peptide more resistant to enzymatic hydrolysis, e.g. as described in WO99/46283.

When present, Z represents a peptide sequence of 1-20 amino acid residues, e.g. in the range of 1-15, more preferably in the range of 1-10, in particular in the range of 1-7 amino acid residues, e.g., 1, 2, 3, 4, 5, 6 or 7 amino acid residues, such as 6 amino acid residues. Each of the amino acid residues in the peptide sequence Z may independently be selected from Ala, Leu, Ser, Thr, Tyr, Cys, Glu, Lys, Arg, Dbu (2,4 diaminobutyric acid), Dpr (2,3-diaminopropanoic acid) and Orn (ornithine). Preferably, the amino acid residues are selected from Ser, Thr, Tyr, Glu, Lys, Arg, Dbu, Dpr and Orn, more preferably selected exclusively from Glu, Lys, and Cys. The above-mentioned amino acids may have either D- or L-configuration, but preferably have an L-configuration.

Particularly preferred sequences Z are sequences of three, four, five, six or seven consecutive lysine residues (i.e. Lys₃, Lys₄, Lys₅, Lys₆ or Lys₇), and particularly five or six consecutive lysine residues. Other exemplary sequences of Z are shown in WO 01/04156. Alternatively the C-terminal residue of the sequence Z may be a Cys residue. This may assist in modification (e.g. PEGylation, or conjugation to albumin) of the compound. In such embodiments, the sequence Z may, for example, be only one amino acid in length (i.e. Z=Cys) or may be two, three, four, five, six or even more amino acids in length. The other amino acids therefore serve as a spacer between the peptide X and the terminal Cys residue.

The peptide sequence Z has no more than 25% sequence identity with the corresponding sequence of the IP-1 portion of human OXM (which has the sequence Lys-Arg-Asn-Arg-Asn-Asn-Ile-Ala).

“Percent (%) amino acid sequence identity” of a given peptide or polypeptide sequence with respect to another polypeptide sequence (e.g. IP-1) is calculated as the percentage of amino acid residues in the given peptide sequence that are identical with correspondingly positioned amino acid residues in the corresponding sequence of that other polypeptide when the two are aligned with one another, introducing gaps for optimal alignment if necessary. % identity values may be determined using WU-BLAST-2 (Altschul et al., Methods in Enzymology, 266:460-480 (1996)). WU-BLAST-2 uses several search parameters, most of which are set to the default values. The adjustable parameters are set with the following values: overlap span=1, overlap fraction=0.125, word threshold (T)=11. A % amino acid sequence identity value is determined by the number of matching identical residues as determined by WU-BLAST-2, divided by the total number of residues of the reference sequence (gaps introduced by WU-BLAST-2 into the reference sequence to maximize the alignment score being ignored), multiplied by 100.

Thus, when Z is aligned optimally with the 8 amino acids of IP-1, it has no more than two amino acids which are identical with the corresponding amino acids of IP-1.

In some embodiments, Z is absent in the compound of the invention.

One or more of the side chains in amino acid residue(s) Y of peptide X is conjugated to a lipophilic substituent Z¹ or Z¹Z². Thus the lipophilic substituent Z¹ may be covalently bonded directly to an atom in the amino acid side chain, or alternatively may comprise a lipophilic moiety Z¹ conjugated to the amino acid side chain by a spacer Z². A lipophilic substituent Z¹ or Z¹Z² may additionally be conjugated to a side chain of an amino acid which is part of the peptide Z if desired.

Without wishing to be bound by any particular theory, it is thought that the lipophilic substituent binds albumin in the blood stream, thus shielding the compounds of the invention from enzymatic degradation and thereby enhancing the half-life of the compounds. The spacer, when present, is used to provide spacing between the compound and the lipophilic substituent.

The lipophilic substituent (or moiety, as appropriate) may be attached to the amino acid side chain or to the spacer via an ester, a sulphonyl ester, a thioester, an amide or a sulphonamide. Accordingly it will be understood that preferably the lipophilic substituent includes an acyl group, a sulphonyl group, an N atom, an O atom or an S atom which forms part of the ester, sulphonyl ester, thioester, amide or sulphonamide. Preferably, an acyl group in the lipophilic substituent forms part of an amide or ester with the amino acid side chain or the spacer.

The lipophilic substituent (or moiety) may be or may include a hydrocarbon chain having 4 to 30 C atoms. Preferably it has at least 8 or 12 C atoms, and preferably it has 24 C atoms or fewer, or 20 C atoms or fewer. The hydrocarbon chain may be linear or branched and may be saturated or unsaturated. It will be understood that the hydrocarbon chain is preferably substituted with a moiety which forms part of the attachment to the amino acid side chain or the spacer, for example an acyl group, a sulphonyl group, an N atom, an O atom or an S atom. Most preferably the hydrocarbon chain is substituted with acyl, and accordingly the hydrocarbon chain may be part of an alkanoyl group, for example decanoyl (caproyl), dodecanoyl (lauroyl), tetradecanoyl (myristoyl), hexadecanoyl (palmitoyl), heptadecanoyl, octadecanoyl (stearoyl), eicosanoyl or docosanoyl.

Thus, the or each Z¹ may be, or may comprise, a decanoyl (caproyl), dodecanoyl (lauroyl), tetradecanoyl (myristoyl), hexadecanoyl (palmitoyl), heptadecanoyl, octadecanoyl (stearoyl), eicosanoyl or docosanoyl group.

Accordingly, the lipophilic substituent may have the formula shown below:

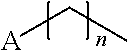

A may be, for example, an acyl group, a sulphonyl group, NH, N-alkyl, an O atom or an S atom, preferably acyl. n is an integer from 3 to 29, preferably at least 7 or at least 11, and preferably 23 or less, more preferably 19 or less.

The hydrocarbon chain may be further substituted. For example, it may be further substituted with up to three substituents selected from NH₂, OH and COOH. If the hydrocarbon chain is further substituted, preferably it is further substituted with only one substituent. Alternatively or additionally, the hydrocarbon chain may include a cycloalkane or heterocycloalkane, for example as shown below:

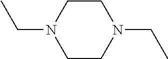

Preferably the cycloalkane or heterocycloalkane is a six-membered ring. Most preferably, it is piperidine.

Alternatively, the lipophilic substituent may be based on a cyclopentanophenanthrene skeleton, which may be partially or fully unsaturated, or saturated. The carbon atoms in the skeleton each may be substituted with Me or OH. For example, the lipophilic substituent may be cholyl, deoxycholyl or lithocholyl.

As mentioned above, the lipophilic substituent may be conjugated to the amino acid side chain by a spacer. When present, the spacer is attached to the lipophilic substituent and to the amino acid side chain. The spacer may be attached to the lipophilic substituent and to the amino acid side chain independently by an ester, a sulphonyl ester, a thioester, an amide or a sulphonamide. Accordingly, it may include two moieties independently selected from acyl, sulphonyl, an N atom, an O atom or an S atom. The spacer may have the formula:

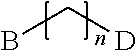

wherein B and D are each independently selected from acyl, sulphonyl, NH, N-alkyl, an O atom and an S atom, preferably from acyl and NH. Preferably, n is an integer from 1 to 10, preferably from 1 to 5. The spacer may be further substituted with one or more substituents selected from C₀₋₆ alkyl, C₀₋₆ alkyl amine, C₀₋₆ alkyl hydroxy and C₀₋₆ alkyl carboxy.

Alternatively, the spacer may have two or more repeat units of the formula above. B, D and n are each selected independently for each repeat unit. Adjacent repeat units may be covalently attached to each other via their respective B and D moieties. For example, the B and D moieties of the adjacent repeat units may together form an ester, a sulphonyl ester, a thioester, an amide or a sulphonamide. The free B and D units at each end of the spacer are attached to the amino acid side chain and the lipophilic substituent as described above.

Preferably the spacer has five or fewer, four or fewer or three or fewer repeat units. Most preferably the spacer has two repeat units, or is a single unit.

The spacer (or one or more of the repeat units of the spacer, if it has repeat units) may be, for example, a natural or unnatural amino acid. It will be understood that for amino acids having functionalised side chains, B and/or D may be a moiety within the side chain of the amino acid. The spacer may be any naturally occurring or unnatural amino acid. For example, the spacer (or one or more of the repeat units of the spacer, if it has repeat units) may be Gly, Pro, Ala, Val, Leu, Ile, Met, Cys, Phe, Tyr, Trp, His, Lys, Arg, Gln, Asn, α-Glu, γ-Glu, Asp, Ser, Thr, Gaba, Aib, β-Ala, 5-aminopentanoyl, 6-aminohexanoyl, 7-aminoheptanoyl, 8-aminooctanoyl, 9-aminononanoyl or 10-aminodecanoyl.

For example, the spacer may be a single amino acid selected from γ-Glu, Gaba, β-Ala and α-Glu.

The lipophilic substituent is conjugated to an amino acid side chain of a Lys, Cys or Orn residue. Preferably, the lipophilic substituent is conjugated to Lys.

An example of a lipophilic substituent and spacer is shown in the formula below:

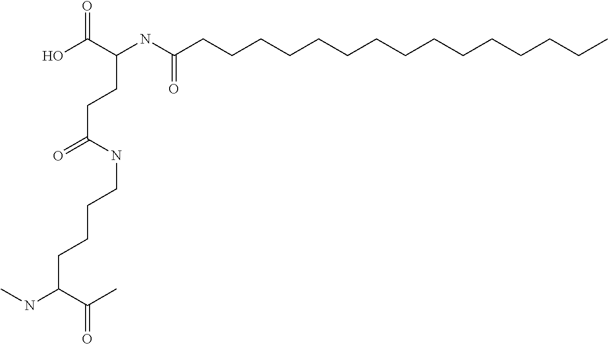 [SMILES: CCCCCCCCCCCCCCCC(=O)NC(CCC(=O)NCCCCC(NC)C(C)=O)C(=O)O]

which is hexadecanoyl-iso-glutamic acid.

Here, a Lys residue in the compound of the present invention is covalently attached to γ-Glu (the spacer) via an amide moiety. Hexadecanoyl (palmitoyl) is covalently attached to the γ-Glu spacer via an amide moiety.

Alternatively or additionally, one or more amino acid side chains in the compound of the invention may be conjugated to a polymeric moiety, for example, in order to increase solubility and/or half-life in vivo (e.g. in plasma) and/or bioavailability. Such modification is also known to reduce clearance (e.g. renal clearance) of therapeutic proteins and peptides.

The polymeric moiety is preferably water-soluble (amphiphilic or hydrophilic), non-toxic, and pharmaceutically inert. Suitable polymeric moieties include polyethylene glycol (PEG), homo- or co-polymers of PEG, a monomethyl-substituted polymer of PEG (mPEG), and polyoxyethylene glycerol (POG). See, for example, Int. J. Hematology 68:1 (1998); Bioconjugate Chem. 6:150 (1995); and Crit. Rev. Therap. Drug Carrier Sys. 9:249 (1992).

Other suitable polymeric moieties include poly-amino acids such as poly-lysine, poly-aspartic acid and poly-glutamic acid (see for example Gombotz, et al. (1995), Bioconjugate Chem., vol. 6: 332-351; Hudecz, et al. (1992), Bioconjugate Chem., vol. 3, 49-57; Tsukada, et al. (1984), J. Natl. Cancer Inst., vol 73: 721-729; and Pratesi, et al. (1985), Br. J. Cancer, vol. 52: 841-848).

The polymeric moiety may be straight-chain or branched. It may have a molecular weight of 500-40,000 Da, for example 500-10,000 Da, 1000-5000 Da, 10,000-20,000 Da, or 20,000-40,000 Da.

A compound of the invention may comprise two or more such moieties, in which case the total molecular weight of all such moieties will generally fall within the ranges provided above.

The polymeric moiety may be coupled (by covalent linkage) to an amino, carboxyl or thiol group of an amino acid side chain. Preferred examples are the thiol group of Cys residues and the epsilon amino group of Lys residues. The carboxyl groups of Asp and Glu residues may also be used.

The skilled reader will be well aware of suitable techniques that can be used to perform the coupling reaction. For example, a PEG moiety carrying a methoxy group can be coupled to a Cys thiol group by a maleimido linkage using reagents commercially available from Nektar Therapeutics AL. See also WO 2008/101017, and the references cited above, for details of suitable chemistry.

In another aspect, one or more of the amino acid side chains in a compound in the present invention, for example in peptide X, is/are conjugated to a polymeric moiety.

In a further aspect, the present invention provides a composition comprising a compound of the invention as described herein, or a salt or derivative thereof, in admixture with a carrier.

The term “derivative thereof” refers to a derivative of any one of the compounds. Derivatives include all chemical modifications, all conservative variants, all prodrugs and all metabolites of the compounds.

The invention also provides the use of a compound of the present invention in the preparation of a medicament for the treatment of a condition as described below.

The invention also provides a composition wherein the composition is a pharmaceutically acceptable composition, and the carrier is a pharmaceutically acceptable carrier.

### Peptide Synthesis

The compounds of the present invention may be manufactured either by standard synthetic methods, recombinant expression systems, or any other state of the art method. Thus the glucagon analogues may be synthesized in a number of ways, including, for example, a method which comprises:

(a) synthesizing the peptide by means of solid-phase or liquid-phase methodology, either stepwise or by fragment assembly, and isolation and purifying of the final peptide product; or
(b) expressing a nucleic acid construct that encodes the peptide X—Z (i.e. the peptide backbone of the compound of the invention) in a host cell, and recovering the expression product from the host cell culture; or
(c) effecting cell-free in vitro expression of a nucleic acid construct that encodes the peptide X—Z (i.e. the peptide backbone of the compound of the invention), and recovering the expression product;
or any combination of methods of (a), (b), and (c) to obtain fragments of the peptide, subsequently ligating the fragments to obtain the peptide, and recovering the peptide. Typically, methods (b) and (c) will be supplemented by adding the lipophilic substituent at the appropriate location within the peptide backbone after synthesis. In method (a), the derivatised amino acid may be incorporated directly during synthesis, or the lipophilic substituent may be added subsequently.

It is preferred to synthesize the analogues of the invention by means of solid-phase or liquid-phase peptide synthesis. In this context, reference is made to WO 98/11125 and, among many others, Fields, G B et al., 2002, “Principles and practice of solid-phase peptide synthesis”. In: Synthetic Peptides (2nd Edition), and the Examples herein.

For recombinant expression, the nucleic acid fragments of the invention will normally be inserted in suitable vectors to form cloning or expression vectors carrying the nucleic acid fragments of the invention; such novel vectors are also part of the invention. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) of the invention are capable of autonomous replication, thereby enabling high copy-numbers for the purposes of high-level expression or high-level replication for subsequent cloning.

In general outline, an expression vector comprises the following features in the 5′→3′ direction and in operable linkage: a promoter for driving expression of the nucleic acid fragment of the invention, optionally a nucleic acid sequence encoding a leader peptide enabling secretion (to the extracellular phase or, where applicable, into the periplasma), the nucleic acid fragment encoding the peptide of the invention, and optionally a nucleic acid sequence encoding a terminator. They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

The vectors of the invention are used to transform host cells to produce the compound of the invention. Such transformed cells, which are also part of the invention, can be cultured cells or cell lines used for propagation of the nucleic acid fragments and vectors of the invention, or used for recombinant production of the peptides of the invention.

Preferred transformed cells of the invention are micro-organisms such as bacteria [such as the species Escherichia (e.g. E. coli), Bacillus (e.g. Bacillus subtilis), Salmonella, or Mycobacterium (preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment are useful embodiments of the invention; they can be used for small-scale or large-scale preparation of the peptides of the invention.

When producing the peptide of the invention by means of transformed cells, it is convenient, although far from essential, that the expression product is secreted into the culture medium.

It will be understood that recombinatn expression of X—Z may only be appropriate when each residue in X—Z is one of the 20 naturally occurring amino acids which can be incorporated into protein by conventional nucleic acid translation. However, modified translation systems are known which can introduce non-conventional amino acids and such systems may be used if desired.

An exemplary synthesis route for a compound of the invention is illustrated below. A person skilled in the art will be able to adapt the shown procedure as required in order to optimise the process for any compound of choice.

### Efficacy

Binding of the relevant compounds to GLP-1 or glucagon (Glu) receptors may be used as an indication of agonist activity, but in general it is preferred to use a biological assay which measures intracellular signalling caused by binding of the compound to the relevant receptor. For example, activation of the glucagon receptor by a glucagon agonist will stimulate cellular cyclic AMP (cAMP) formation. Similarly, activation of the GLP-1 receptor by a GLP-1 agonist will stimulate cellular cAMP formation. Thus, production of cAMP in suitable cells expressing one of these two receptors can be used to monitor the relevant receptor activity. Use of a suitable pair of cell types, each expressing one receptor but not the other, can hence be used to determine agonist activity towards both types of receptor.

The skilled person will be aware of suitable assay formats, and examples are provided below. The GLP-1 receptor and/or the glucagon receptor may have the sequence of the receptors as described in the examples. For example, the assays may employ the human glucagon receptor (Glucagon-R) having primary accession number GI:4503947 and/or the human glucagon-like peptide 1 receptor (GLP-1R) having primary accession number GI:166795283. (in that where sequences of precursor proteins are referred to, it should of course be understood that assays may make use of the mature protein, lacking the signal sequence).

EC₅₀ values may be used as a numerical measure of agonist potency at a given receptor. An EC₅₀ value is a measure of the concentration of a compound required to achieve half of that compound's maximal activity in a particular assay. Thus, for example, a compound having EC₅₀[GLP-1] lower than the EC₅₀[GLP-1] of glucagon in a particular assay may be considered to have higher GLP-1 receptor agonist potency than glucagon.

The compounds described in this specification are typically Glu-GLP-1 dual agonists, as determined by the observation that they are capable of stimulating cAMP formation at both the glucagon receptor and the GLP-1 receptor. The stimulation of each receptor can be measured in independent assays and afterwards compared to each other.

By comparing the EC₅₀ value for the glucagon receptor (EC₅₀ [Glucagon-R]) with the EC₅₀ value for the GLP-1 receptor, (EC₅₀ [GLP-1R]) for a given compound, the relative glucagon selectivity (%) of that compound can be found as follows:

Relative Glucagon-R selectivity[compound]=(1/EC₅₀[Glucagon-R])×100/(1/EC₅₀[Glucagon-R]+1/EC₅₀[GLP-1R])

The relative GLP-1R selectivity can likewise be found:

Relative GLP-1R selectivity[compound]=(1/EC₅₀[GLP-1R])×100/(1/EC₅₀[Glucagon-R]+1/EC₅₀[GLP-1R])

A compound's relative selectivity allows its effect on the GLP-1 or glucagon receptor to be compared directly to its effect on the other receptor. For example, the higher a compound's relative GLP-1 selectivity is, the more effective that compound is on the GLP-1 receptor as compared to the glucagon receptor.

Using the assays described below, we have found the relative GLP-1 selectivity for human glucagon to be approximately 5%.

The compounds of the invention have a higher relative GLP-1R selectivity than human glucagon in that for a particular level of glucagon-R agonist activity, the compound will display a higher level of GLP-1R agonist activity (i.e. greater potency at the GLP-1 receptor) than glucagon. It will be understood that the absolute potency of a particular compound at the glucagon and GLP-1 receptors may be higher, lower or approximately equal to that of native human glucagon, as long as the appropriate relative GLP-1R selectivity is achieved.

Nevertheless, the compounds of this invention may have a lower EC₅₀ [GLP-1R] than human glucagon. The compounds may have a lower EC₅₀[GLP-1-R] than glucagon while maintaining an EC₅₀ [Glucagon-R] that is less than 10-fold higher than that of human glucagon, less than 5-fold higher than that of human glucagon, or less than 2-fold higher than that of human glucagon.

The compounds of the invention may have an EC₅₀ [Glucagon-R] that is less than two-fold that of human glucagon. The compounds may have an EC₅₀ [Glucagon-R] that is less than two-fold that of human glucagon and have an EC₅₀ [GLP-1R] that is less than half that of human glucagon, less than a fifth of that of human glucagon, or less than a tenth of that of human glucagon.

The relative GLP-1R selectivity of the compounds may be between 5% and 95%. For example, the compounds may have a relative selectivity of 5-20%, 10-30%, 20-50%, 30-70%, or 50-80%; or of 30-50%, 40-60,%, 50-70% or 75-95%.

The compounds of the invention may also have effect on other Class B GPCR receptors, such as, but not limited to, Calcitonin gene-related peptide 1 (CGRP1), corticotropin-releasing factor 1 & 2 (CRF1 & CRF2), gastric inhibitory polypeptide (GIP), glucagon-like peptide 1 & 2 (GLP-1 & GLP-2, glucagon (GCGR), secretin, gonadotropin releasing hormone (GnRH), parathyroid-hormone 1 & 2 (PTH1 & PTH2), vasoactive intestinal peptide (VPAC1 & VPAC2).

### Therapeutic Uses

The compounds of the invention may provide an attractive treatment option for, inter alia metabolic diseases, including, obesity, dyslipidemia and diabetes mellitus (diabetes).

Metabolic syndrome is characterized by a group of metabolic risk factors in one person. They include abdominal obesity (excessive fat tissue around the abdominal internal organs), atherogenic dyslipidemia (blood fat disorders including high triglycerides, low HDL cholesterol and/or high LDL cholesterol, which foster plaque buildup in artery walls), elevated blood pressure (hypertension), insulin resistance and glucose intolerance, prothrombotic state (e.g. high fibrinogen or plasminogen activator inhibitor-1 in the blood), and proinflammatory state (e.g., elevated C-reactive protein in the blood).

Individuals with the metabolic syndrome are at increased risk of coronary heart disease and other diseases related to other manifestations of arteriosclerosis (e.g., stroke and peripheral vascular disease). The dominant underlying risk factors for this syndrome appear to be abdominal obesity.

Diabetes comprises a group of metabolic diseases characterized by hyperglycemia resulting from defects in insulin secretion, insulin action, or both. Acute signs of diabetes include excessive urine production, resulting compensatory thirst and increased fluid intake, blurred vision, unexplained weight loss, lethargy, and changes in energy metabolism. The chronic hyperglycemia of diabetes is associated with long-term damage, dysfunction, and failure of various organs, notably the eyes, kidneys, nerves, heart and blood vessels. Diabetes is classified into type 1 diabetes, type 2 diabetes and gestational diabetes on the basis on pathogenetic characteristics.

Type 1 diabetes accounts for 5-10% of all diabetes cases and is caused by auto-immune destruction of insulin-secreting pancreatic β-cells.

Type 2 diabetes accounts for 90-95% of diabetes cases and is a result of a complex set of metabolic disorders. Type 2 diabetes is the consequence of endogenous insulin production becoming insufficient to maintain plasma glucose levels below the diagnostic thresholds.

Gestational diabetes refers to any degree of glucose intolerance identified during pregnancy.

Pre-diabetes includes impaired fasting glucose and impaired glucose tolerance and refers to those states that occur when blood glucose levels are elevated but below the levels that are established for the clinical diagnosis for diabetes.

A large proportion of people with type 2 diabetes and pre-diabetes are at increased risk of morbidity and mortality due to the high prevalence of additional metabolic risk factors including abdominal obesity (excessive fat tissue around the abdominal internal organs), atherogenic dyslipidemia (blood fat disorders including high triglycerides, low HDL cholesterol and/or high LDL cholesterol, which foster plaque buildup in artery walls), elevated blood pressure (hypertension) a prothrombotic state (e.g. high fibrinogen or plasminogen activator inhibitor-1 in the blood), and proinflammatory state (e.g., elevated C-reactive protein in the blood).

Conversely, obesity confers an increased risk of developing pre-diabetes, type 2 diabetes as well as e.g. certain types of cancer, obstructive sleep apnea and gall-blader disease.

Dyslipidaemia is associated with increased risk of cardiovascular disease. High Density Lipoprotein (HDL) is of clinical importance since an inverse correlation exists between plasma HDL concentrations and risk of atherosclerotic disease. The majority of cholesterol stored in atherosclerotic plaques originates from LDL and hence elevated concentrations Low Density Lipoproteins (LDL) is closely associated with atherosclerosis. The HDL/LDL ratio is a clinical risk indictor for atherosclerosis and coronary atherosclerosis in particular.

Without wishing to be bound by any particular theory, it is believed that the compounds of the invention act as GluGLP-1 dual agonists. The dual agonist combines the effect of glucagon on fat metabolism with the effects of GLP-1 on food intake. They might therefore act in a synergistic fashion to accelerate elimination of excessive fat deposition and induce sustainable weight loss. Certain of the compounds described may also have a beneficial effect on glucose control directly, independently of any effect on body weight.

The synergistic effect of dual GluGLP-1 agonists may also result in reduction of cardiovascular risk factors such as high cholesterol and LDL, which may be entirely independent of their effect on body weight.

The compounds of the present invention may therefore be used as pharmaceutical agents for preventing weight gain, promoting weight loss, reducing excess body weight or treating obesity (e.g. by control of appetite, feeding, food intake, calorie intake, and/or energy expenditure), including morbid obesity, as well as associated diseases and health conditions caused or characterised by excess body weight. These include but are not limited to obesity, morbid obesity, morbid obesity prior to surgery, obesity linked inflammation, obesity linked gallbladder disease and obesity induced sleep apnea. The compounds of the invention may also be used for treatment of metabolic syndrome, hypertension, type II diabetes, atherogenic dyslipidemia, atherosclerois, arteriosclerosis, coronary heart disease, peripheral artery disease, stroke and microvascular disease. These are all conditions which can be associated with obesity. However, the effects of the compounds of the invention on these conditions may be mediated in whole or in part via an effect on body weight, or may be independent thereof. Further, via their direct effect on glucose control, the compounds of the present invention may be useful for treatment of any of the above conditions as well as others not necessarily associated with or caused by excess body weight, including type I diabetes and gestational diabetes.

The compounds of the present invention may further be used as pharmaceutical agents for lowering circulating LDL levels, and/or increasing HDL/LDL ratio.

### Combination Therapy

As noted above, it will be understood that reference in the following to a compound of the invention also extends to a pharmaceutically acceptable salt or solvate thereof as well as to a composition comprising more than one different compounds of the invention.

A compound of the invention may be administered as part of a combination therapy with an agent for treatment of obesity, hypertension dyslipidemia or diabetes.

In such cases, the two active agents may be given together or separately, and as part of the same pharmaceutical formulation or as separate formulations.

Thus a compound or salt thereof can further be used in combination with an anti-obesity agent, including but not limited to a glucagon-like peptide receptor 1 agonist, peptide YY or analogue thereof, cannabinoid receptor 1 antagonist, lipase inhibitor, melanocortin receptor 4 agonist, or melanin concentrating hormone receptor 1 antagonist.

A compound of the invention or salt thereof can be used in combination with an anti-hypertension agent, including but not limited to an angiotensin-converting enzyme inhibitor, angiotensin II receptor blocker, diuretics, beta-blocker, or calcium channel blocker.

A compound of the invention or salt thereof can be used in combination with a dyslipidaemia agent, including but not limited to a statin, a fibrate, a niacin and/or a cholesterol absorbtion inhibitor.

Further, a compound of the invention or salt thereof can be used in combination with an anti-diabetic agent, including but not limited to metformin, a sulfonylurea, a glinide, a DPP-IV inhibitor, a glitazone, a different GLP-1 agonist or an insulin. In a preferred embodiment, the compound or salt thereof is used in combination with insulin, DPP-IV inhibitor, sulfonylurea or metformin, particularly sulfonylurea or metformin, for achieving adequate glycemic control. In an even more preferred embodiment the compound or salt thereof is used in combination with an insulin or an insulin analogue for achieving adequate glycemic control. Examples of insulin analogues include but are not limited to Lantus, Novorapid, Humalog, Novomix, and Actraphane HM.

### Pharmaceutical Compositions

The compounds of the present invention, or salts thereof, may be formulated as pharmaceutical compositions prepared for storage or administration, which typically comprise a therapeutically effective amount of a compound of the invention, or a salt thereof, in a pharmaceutically acceptable carrier.

Such compositions comprise those of overall solid form as well as those of overall pasteous or liquid form, which can be selected and optimised with respect to the specific route of administration and/or needs of the patient. Such forms are per se known to a person skilled in the art.

The therapeutically effective amount of a compound of the present invention will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. pH buffering agents may be phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, which is a preferred buffer, arginine, lysine, or acetate or mixtures thereof. The term further encompases any agents listed in the US Pharmacopeia for use in animals, including humans.

The term “pharmaceutically acceptable salt” refers to a salt of any one of the compounds. Salts include pharmaceutically acceptable salts such as acid addition salts and basic salts. Examples of acid addition salts include hydrochloride salts, citrate salts and acetate salts. Examples of basic salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17th edition. Ed. Alfonso R. Gennaro (Ed.), Mark Publishing Company, Easton, Pa., U.S.A., 1985 and more recent editions, and in the Encyclopaedia of Pharmaceutical Technology.

“Treatment” is an approach for obtaining beneficial or desired clinical results. For the purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. In certain embodiments, packaged forms include a label or insert with instructions for use. Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, and transdermal) administration. The formulations may conveniently be presented in unit dosage form and may be prepared by any of the methods well known in the art of pharmacy.

Subcutaneous or transdermal modes of administration may be particularly suitable for the compounds described herein.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

### Methods

### General Synthesis of Glucagon Analogues

Solid phase peptide synthesis (SPPS) was performed on a microwave assisted synthesizer using standard Fmoc strategy in NMP on a polystyrene resin (TentaGel S Ram). HATU was used as coupling reagent together with DIPEA as base. Piperidine (20% in NMP) was used for deprotection.

Pseudoprolines: Fmoc-Phe-Thr(.Psi. Me, Me pro)-OH and Fmoc-Asp-Ser(.Psi., Me, Me pro)-OH (purchased from NovaBiochem) were used where applicable.

Abbreviations employed are as follows:
ivDde: 1-(4,4-dimethyl-2,6-dioxocyclohexylidene)3-methyl-butyl
Dde: 1-(4,4-dimethyl-2,6-dioxocyclohexylidene)-ethyl
DCM: dichloromethane
DMF: N,N-dimethylformamide
DIPEA: diisopropylethylamine
EtOH: ethanol
Et₂O: diethyl ether
HATU: N—[(dimethylamino)-1H-1,2,3-triazol[4,5-b]pyridine-1-ylmethylene]-N-methylmethanaminium hexafluorophosphate N-oxide
MeCN: acetonitrile
NMP: N-methylpyrrolidone
TFA: trifluoroacetic acid
TIS: triisopropylsilane

### Cleavage:

The crude peptide was cleaved from the resin by treatment with 95/2.5/2.5% (v/v) TFA/TIS/water at r.t. for 2 h. For peptides with a methionine in the sequence a mixture of 95/5% (v/v) TFA/EDT was used. Most of the TFA was removed at reduced pressure and the crude peptide was precipitated and washed with diethylether and allowed to dry to constant weight at ambient temperature.

### General Synthesis of Acylated Glucagon Analogues

The peptide backbone was synthesized as described above for the general synthesis of glucagon analogues, with the exception that it was acylated on the side chain of a lysine residue with the peptide still attached to the resin and fully protected on the side chain groups, except the epsilon-amine on the lysine to be acylated. The lysine to be acylated was incorporated with the use of Fmoc-Lys(ivDde)-OH or Fmoc-Lys(Dde)-OH. The N-terminal of the peptide was protected with a Boc group using Boc₂O in NMP. While the peptide was still attached to the resin, the ivDde protecting group was selectively cleaved using 5% hydrazine hydrate in NMP. The unprotected lysine side chain was then first coupled with a spacer amino acid like Fmoc-Glu-OtBu, which was deprotected with piperidine and acylated with a fatty acid using standard peptide coupling methodology as described above. Alternatively, the histidine at the N-terminal may be incorporated from the beginning as Boc-His(Boc)-OH. Cleavage from the resin and purification were performed as described above.

An alternative strategy is to use Fmoc-Lys(Hexadecanoyl-isoGlu(tBu))-OH for easy incorporation of the fatty acid and linker as part of the standard synthesis procedure.

### Generation of Cell Lines Expressing Human Glucagon- and GLP-1 Receptors

The cDNA encoding either the human glucagon receptor (Glucagon-R) (primary accession number P47871) or the human glucagon-like peptide 1 receptor (GLP-1R) (primary accession number P43220) were cloned from the cDNA clones BC104854 (MGC:132514/IMAGE:8143857) or BC112126 (MGC:138331/IMAGE:8327594), respectively. The DNA encoding the Glucagon-R or the GLP-1-R was amplified by PCR using primers encoding terminal restriction sites for subcloning. The 5′-end primers additionally encoded a near Kozak consensus sequence to ensure efficient translation. The fidelity of the DNA encoding the Glucagon-R and the GLP-1-R was confirmed by DNA sequencing. The PCR products encoding the Glucagon-R or the GLP-1-R were subcloned into a mammalian expression vector containing a neomycin (G418) resistance marker.

The mammalian expression vectors encoding the Glucagon-R or the GLP-1-R were transfected into HEK293 cells by a standard calcium phosphate transfection method. 48 hr after transfection cells were seeded for limited dilution cloning and selected with 1 mg/ml G418 in the culture medium. Three weeks later 12 surviving colonies of Glucagon-R and GLP-1-R expressing cells were picked, propagated and tested in the Glucagon-R and GLP-1-R efficacy assays as described below. One Glucagon-R expressing clone and one GLP-1-R expressing clone were chosen for compound profiling.

### Glucagon Receptor and GLP-1-Receptor Efficacy Assays

HEK293 cells expressing the human Glucagon-R, or human GLP-1-R were seeded at 40,000 cells per well in 96-well microtiter plates coated with 0.01% poly-L-lysine and grown for 1 day in culture in 100 μl growth medium. On the day of analysis, growth medium was removed and the cells washed once with 200 μl Tyrode buffer. Cells were incubated in 100 μl Tyrode buffer containing increasing concentrations of test peptides, 100 μM IBMX, and 6 mM glucose for up 15 min at 37° C. The reaction was stopped by addition of 25 μl 0.5 M HCl and incubated on ice for 60 min. The cAMP content was estimated using the FlashPlate® cAMP kit from Perkin-Elmer according to manufacturer instructions. EC₅₀ and relative efficacies compared to reference compounds (glucagon and GLP-1) were estimated by computer aided curve fitting.

### Example 1

### Synthesis of Compound 1

H—H-Aib-QGTFTSDYSKYLD-K(Hexadecanoyl-isoGlu)-RRAKDFIEWLLSA-NH2 (Compound 1)

The peptide was synthesized on a CEM Liberty Peptide Synthesizer using TentaGel S Ram resin (1.04 g; 0.25 mmol/g) and Fmoc chemistry as described above using Fmoc-Phe-Thr(ψ-Me,Me-Pro)-OH and. Fmoc-Lys(Hexadecanoyl-isoGlu(tBu))-OH (Corden Pharma) was coupled manually using 396 mg dissolved in DMF/DCM (2:1, 8 ml) with HATU (190 mg). The solution was added to the resin and then DIEA (86 μl) was added. The resin was shaken gently for 4 hours and then washed with DMF (8×2 min).

The peptide was cleaved from the resin as described above. The crude peptide was purified on a Gemini column (5×25 cm; 10 μm; C18) with a 35 ml/min flow of a mixture of buffer A (0.1% TFA; aq.) and buffer B (0.1% TFA; 90% MeCN; aq.). The product was eluted with a linear gradient from 20% to 70% buffer B over 47 min, and fractions (9 ml) were collected with a fraction collector. Relevant fractions were analysed by analytical HPLC and MS, pooled and lyophilised to give a white powder (88 mg; 95%). The mass was 3826.03 Da as determined by MS (Calc. 3826.05 Da).

### Example 2

### Activity at Glucagon and GLP-1 Receptors

TABLE 1
EC50 values for cAMP generation in HEK293 cells
expressing GLP-1 receptor or Glucagon receptor

EC50 (nM)

| Compound | Glucagon receptor | GLP-1 receptor |
|---|---|---|
| 1 | 0.15 | 0.12 |
| 2 | 0.35 | 0.36 |
| 4 | 0.79 | 1.31 |
| 7 | 0.06 | 0.06 |
| 8 | 0.20 | 0.20 |
| 9 | 0.10 | 0.05 |
| 10 | 0.06 | 0.47 |
| 11 | 0.09 | 0.15 |
| 12 | 0.14 | 0.06 |
| 14 | 0.19 | 0.12 |
| 15 | 0.42 | 0.06 |
| 16 | 0.11 | 0.06 |
| 17 | 0.05 | 0.07 |
| 18 | 0.09 | 0.09 |
| 21 | 0.21 | 0.08 |

1-4. (canceled)

5. A compound having the formula
R¹—X—Z—R²
wherein
R¹ is H, C₁₋₄ alkyl, acetyl, formyl, benzoyl or trifluoroacetyl;
R² is OH or NH₂;
X is a peptide which has the formula II:
(SEQ ID NO: 18)
His-X2-X3-Gly-Thr-Phe-Thr-Ser-Asp-Tyr-Ser-X12-Tyr-Leu-Asp-X16-X17-Arg-Ala-
X20-Asp-Phe-Ile-X24-Trp-Leu-X27-X28-X29 (II)
wherein
X2 is selected from Ser, D-Ser and Aib;
X3 is selected from Gln, His and Pro;
X12 is selected from Arg, Lys and Y;
X16 is selected from Glu and Y;
X17 is selected from Arg and Y;
X20 is selected from Lys, Arg and Y;
X24 is selected from Glu and Y;
X27 is selected from Leu and Y;
X28 is selected from Ser and Y or absent;
X29 is Ala or absent;
wherein X12 and/or X20 is Arg;
wherein at least one of X12, X16, X17, X20, X24, X27 and X28 is Y;
wherein each residue Y is independently selected from Lys, Cys and Orn;
wherein the side chain of at least one amino acid residue Y is conjugated to a lipophilic substituent having the formula:
(i) Z¹, wherein Z¹ is a lipophilic moiety conjugated directly to the side chain of Y; or
(ii) Z¹Z², wherein Z¹ is a lipophilic moiety, Z² is a spacer, and Z¹ is conjugated to the side chain of Y via Z²;
and Z is absent or is a sequence of 1-20 amino acid units independently selected from the group consisting of Ala, Leu, Ser, Thr, Tyr, Cys, Glu, Lys, Arg, Dbu, Dpr and Orn;
or a pharmaceutically acceptable salt thereof.

6. A compound according to claim 5 wherein X12 is Arg.

7. A compound according to claim 5 wherein X has the formula IIa:
(SEQ ID NO: 19)
His-X2-X3-Gly-Thr-Phe-Thr-Ser-Asp-Tyr-Ser-Arg-Tyr-Leu-Asp-X16-X17-Arg-Ala-X20-Asp-Phe-Ile-
X24-Trp-Leu-Leu-Ser-Ala (IIa)
wherein
X2 is selected from Ser, D-Ser and Aib;
X3 is selected from Gln, His and Pro;
X16 is selected from Glu and Y;
X17 is selected from Arg and Y;
X20 is selected from Arg and Lys; and
X24 is selected from Glu and Y.

8. A compound according to claim 7 wherein X has the formula IIb:
(SEQ ID NO: 20)
His-X2-X3-Gly-Thr-Phe-Thr-Ser-Asp-Tyr-Ser-Arg-Tyr-Leu-Asp-Glu-X17-Arg-Ala-
Arg-Asp-Phe-Ile-Glu-Trp-Leu-Leu-Ser-Ala (IIb)
wherein
X2 is selected from Ser, D-Ser and Aib;
X3 is selected from Gln, His and Pro; and
X17 is Y.

9. A compound according to claim 5 wherein X has the sequence:
(SEQ ID NO: 21)
HSQGTFTSDYSRYLDEKRARDFIEWLLSA;
(SEQ ID NO: 22)
H-DSer-QGTFTSDYSRYLDEKRARDFIEWLLSA;
(SEQ ID NO: 23)
H-Aib-QGTFTSDYSRYLDEKRARDFIEWLLSA;
(SEQ ID NO: 24)
HSHGTFTSDYSRYLDEKRARDFIEWLLSA;
(SEQ ID NO: 25)
H-DSer-HGTFTSDYSRYLDEKRARDFIEWLLSA;
(SEQ ID NO: 26)
H-Aib-GTFTSDYSRYLDEKRARDFIEWLLSA;
(SEQ ID NO: 27)
HSPGTFTSDYSRYLDEKRARDFIEWLLSA;
(SEQ ID NO: 28)
H-DSer-PGTFTSDYSRYLDEKRARDFIEWLLSA;
(SEQ ID NO: 29)
H-Aib-PGTFTSDYSRYLDEKRARDFIEWLLSA;
or
(SEQ ID NO: 30)
H-Aib-QGTFTSDYSRYLDEKRAKDFIEWLLSA.

10. A compound according to claim 9 wherein X is:
(SEQ ID NO: 21)
HSQGTFTSDYSRYLDEK*RARDFIEWLLSA;
(SEQ ID NO: 22)
H-DSer-QGTFTSDYSRYLDEK*RARDFIEWLLSA;
(SEQ ID NO: 23)
H-Aib-QGTFTSDYSRYLDEK*RARDFIEWLLSA;
(SEQ ID NO: 24)
HSHGTFTSDYSRYLDEK*RARDFIEWLLSA;
(SEQ ID NO: 25)
H-DSer-HGTFTSDYSRYLDEK*RARDFIEWLLSA;
(SEQ ID NO: 26)
H-Aib-GTFTSDYSRYLDEK*RARDFIEWLLSA;
(SEQ ID NO: 27)
HSPGTFTSDYSRYLDEK*RARDFIEWLLSA;
(SEQ ID NO: 28)
H-DSer-PGIFTSDYSRYLDEK*RARDFIEWLLSA;
(SEQ ID NO: 29)
H-Aib-PGTFTSDYSRYLDEK*RARDFIEWLLSA;
or
(SEQ ID NO: 30)
H-Aib-QGTFTSDYSRYLDEK*RAKDFIEWLLSA;
wherein K* indicates a Lys residue to which the lipophilic substituent is conjugated.

11-15. (canceled)

16. A compound according to claim 5 wherein peptide X contains only one residue Y.

17. A compound according to claim 5 wherein the or each residue Y is Lys.

18. A compound according to claim 5 wherein Z is selected from Lys₃, Lys₄, Lys₅, Lys₆ and Lys₇.

19. A compound according to claim 5 wherein Z is absent.

20. A compound according to claim 5 wherein the or each Z¹ comprises a hexadecanoyl or octadecanoyl moiety.

21. A compound according to claim 20 wherein the or each lipophilic substituent is hexadecanoyl-isoGlu or octadecanoyl-iso-Glu.

22. A compound according to claim 5 wherein R¹ is H.

23. A compound according to claim 5 wherein R² is NH₂.

24. A compound according to claim 5 wherein one or more of the amino acid side chains in the compound is conjugated to a polymeric moiety.

25. A compound according to claim 24 wherein one or more of the amino acid side chains in peptide X is conjugated to a polymeric moiety.

26. (canceled)

27. A compound according to claim 5 which is:
(SEQ ID NO: 31)
H-HSQGTFTSDYSRYLDE-K(Hexadecanoyl-isoGlu)-RARDFIEWLLSA-NH₂ [Compound 7];
(SEQ ID NO: 32)
H-H-DSer-QGTFTSDYSRYLDE-K(Hexadecanoyl-isoGlu)-RARDFIEWLLSA-NH₂ [Compound 8];
(SEQ ID NO: 33)
H-H-Aib-QGTFTSDYSRYLDE-K(Hexadecanoyl-isoGlu)-RARDFIEWLLSA-NH₂ [Compound 9];
(SEQ ID NO: 34)
H-HSHGTFTSDYSRYLDE-K(Hexadecanoyl-isoGlu)-RARDFIEWLLSA-NH₂ [Compound 10];
(SEQ ID NO: 35)
H-H-DSer-HGTFTSDYSRYLDE-K(Hexadecanoyl-isoGlu)-RARDFIEWLLSA-NH₂ [Compound 11];
(SEQ ID NO: 36)
H-H-Aib-HGTFTSDYSRYLDE-K(Hexadecanoyl-isoGlu)-RARDFIEWLLSA-NH₂ [Compound 12];
(SEQ ID NO: 37)
H-HSPGTFTSDYSRYLDE-K(Hexadecanoyl-isoGlu)-RARDFIEWLLSA-NH₂ [Compound 13];
(SEQ ID NO: 38)
H-H-DSer-PGTFTSDYSRYLDE-K(Hexadecanoyl-isoGlu)-RARDFIEWLLSA-NH₂ [Compound 14];
(SEQ ID NO: 39)
H-H-Aib-PGTFTSDYSRYLDE-K(Hexadecanoyl-isoGlu)-RARDFIEWLLSA-NH₂ [Compound 15];
or
(SEQ ID NO: 40)
H-H-Aib-QGTFTSDYSRYLDE-K(Hexadecanoyl-isoGlu)-RAKDFIEWLLSA-NH₂ [Compound 16];
or a pharmaceutically acceptable salt thereof.

28-29. (canceled)

30. A composition comprising a compound according to claim 5, or a salt or derivative thereof, in admixture with a carrier.

31. A composition according to claim 30 wherein the composition is a pharmaceutically acceptable composition, and the carrier is a pharmaceutically acceptable carrier.

32. An isolated nucleic acid encoding a peptide X—Z as defined in claim 5.

33. A vector comprising a nucleic acid according to claim 32.

34. A host cell comprising a nucleic acid according to claim 32.

35. (canceled)

36. A method of preventing weight gain or promoting weight loss in an individual in need thereof, said method comprising administering to said individual a therapeutically effective amount of a compound according to claim 5.

37. A method of lowering circulating LDL levels, and/or increasing HDL/LDL ratio in an individual in need thereof, said method comprising administering to said individual a therapeutically effective amount of a compound according to claim 5.

38. A method of treatment of a condition caused or characterised by excess body weight in an individual in need thereof, said method comprising administering to said individual a therapeutically effective amount of a compound according to claim 5.

39. A method of prevention and/or treatment of a condition selected from the group consisting of obesity, morbid obesity, morbid obesity prior to surgery, obesity linked inflammation, obesity linked gallbladder disease, obesity induced sleep apnea, type I diabetes, type II diabetes, gestational diabetes, metabolic syndrome, hypertension, atherogenic dyslipidemia, atherosclerois, arteriosclerosis, coronary heart disease, peripheral artery disease, stroke, and microvascular disease in an individual in need thereof, said method comprising administering to said individual a therapeutically effective amount of a compound according to claim 36.

40. The method of claim 39, wherein the compound is administered as part of a combination therapy together with an agent for treatment of obesity.

41. The method of claim 40, wherein the agent for treatment of obesity is selected from the group consisting of a glucagon-like peptide receptor 1 agonist, peptide YY receptor agonist or analogue thereof, cannabinoid receptor 1 antagonist, lipase inhibitor, melanocortin receptor 4 agonist, and melanin concentrating hormone receptor 1 antagonist.

42-44. (canceled)

45. The method of claim 39, wherein the compound is administered as part of a combination therapy together with an agent for treatment of dyslipidemia.

46. The method of claim 45, wherein the agent for treatment of dyslipidaemia is selected from the group consisting of a statin, a fibrate, a niacin, and a cholesterol absorption inhibitor.

47. The method of claim 38, wherein the compound is administered as part of a combination therapy together with an agent for treatment of diabetes.

48. The method of claim 47, wherein the agent for treatment of diabetes is selected from the group consisting of metformin, a sulfonylurea, a glinide, a DPP-IV inhibitor, a glitazone, a GLP-1 agonist, insulin, and an insulin analogue.

49. The method of claim 39, wherein the compound is administered as part of a combination therapy together with an agent for hypertension.

50. The method of claim 49, wherein the agent for treatment of hypertension is selected from the group consisting of an angiotensin-converting enzyme inhibitor, an angiotensin II receptor blocker, a diuretic, a beta-blocker, and a calcium channel blocker.
